(19) United States
(12) Patent Application Publication
(10) Pub. No.: US 2008/0064073 A1
(43) Pub. Date: Mar. 13, 2008

(54) Process for Preparing

(76) Inventors: Claire Tondo Vendruscolo, Pelotas-RS (BR); Angelita da Silveira Moreira, Peloas-RS (BR)

Correspondence Address: MCGLEW & TUTTLE, PC, P.O. BOX 9227, SCARBOROUGH STATION, SCARBOROUGH, NY, 10510-9227, US

(21) Appl. No.: 11/718,780

(22) PCT Filed: Nov. 1, 2005
(86) PCT No.: PCT/BR05/00228

§ 371 Date: Oct. 18, 2007

(30) Foreign Application Priority Data
Nov. 5, 2004 (BR) PI406309-0

(51) Int. Cl. C12P 19/06; C07H 3/00; C12N 1/20

(52) U.S. Cl. 435/104; 435/253.6; 536/123.1

(57) ABSTRACT

A process for obtaining a xantan-like biopolymer from bacterial strains from Xanthomonas arboricola and/or Xanthomonas arboricola pv pruni colonies added to fermentation media containing residual waters and products related to the processing of hull-containing rice and parboilized rice and other nutrients is described, the process starting from an initial pre-inoculum (step 110), yielding a final pre-inoculum (steps 120, 220) and then an inoculum, said inoculum being fermented in a first fermenter under process conditions (steps 130 and 230) and then in a second fermenter (steps 140, 250), then the fermented broth is inactivated and submitted to insolubilization for the recovery of the biopolymer product, step (150), the biopolymer is dried (step 160), and milled or crushed to the desired particle size distribution, (step 170), and the biopolymer is recovered as a powder or an aqueous solution (step 180). Optionally after the second fermentation, (step 240), the fermented broth is centrifuged for cell separation (step 250), and the separated cells are withdrawn or destroyed (step 260a).

The present invention belongs to the field of processes for obtaining biopolymers, more specifically to a process for obtaining xantan-like microbian biopolymers derived from cultures of Xanthomonas arboricola and/or Xanthomonas arboricola pruni bacterial strains.

### BACKGROUND INFORMATION

Microbian biopolymers are polysaccharides obtained with the aid of biotechnological processes through the use of fungi, yeasts or bacteria. The relevance and potential use of biopolymers in wide industrial fields as thickening agents, stabilizers, gellifying agents and emulsifiers in foodstuff, pharmacological products, paints, pesticides, petroleum industry and the like is of common knowledge. Presently, conventional polysaccharides are progressively being replaced by microbia-related products. This is mainly due to the possibility of modifying the rheological features of these products through the control of fermentation parameters, besides independence from climate and batch quality control, among other advantages.

Xantan is a high molecular weight, 2.10⁶ to 12.10⁶ g.mol⁻¹, extracellular anionic polysaccharide, formed by pentasaccharide units repeated from 2,000 to 6,000 times. Xantan is obtained by aerobic fermentation of Xanthomonas campestris, commercially using the campestris patovar. It is formed by the monosaccharides D-manose, D-glycose and D-glycuronic acid, besides pyruvic and acetic radicals.

Xantan's main feature is its ability to modify the rheology or the flow behavior of solutions. Its properties are governed by its chemical composition, structure and molecular links. In spite of being an imported good, Brazil follows the worldwide trend of increasing xantan consumption. Up to the present, in spite of the availability of a diversity of biopolymer-producing bacteria, besides being the main world source of raw materials (saccharose and alcohol) used to produce these biopolymers, Brazil does not manufacture xantan gum.

Bacterial polysaccharides offer the advantages of regular chemical structure, reproducible chemical and physical properties, and constant source of supply since they do not depend on climate conditions to be produced.

The discovery of the xantan biopolymer occurred in the USA in the fifties, caused by the interest in water soluble gums produced by microorganisms, when it could be observed that the Xanthomonas campestris pv campestris NRRL B 1459 strain yielded extremely gummy, viscous colonies.

The first patent document related to xantan production through Xanthomonas campestris pv campestris is U.S. Pat. No. 3,000,790. Many others followed, filed by the US Secretary of Agriculture and companies as Esso Research, Jersey Production Research Co., Kelco Co., Rhone Poulec Industries, Pfizer Inc., Standard Oil Co., Sanofi-Société Nationale Elf Aquitaine on fermentation processes such as U.S. Pat. No. 3,020,206; U.S. Pat. No. 3,251,749; U.S. Pat. No. 3,328,262; U.S. Pat. No. 3,391,060; U.S. Pat. No. 3,391,061; U.S. Pat. No. 3,485,719; FR 2,342,339; FR 2,414,555; U.S. Pat. No. 4,282,321; EP 66,961; EP 66,377; U.S. Pat. No. 4,352,882; U.S. Pat. No. 4,328,310; U.S. Pat. No. 4,400,467; U.S. Pat. No. 4,407,950; U.S. Pat. No. 4,407,951; FR 2,671,097, all of them being related to the use of Xanthomonas campestris; and processes where the inoculum is produced in a medium containing 3 g.L⁻¹ yeast extract, 3 g.L⁻¹ malt extract, 5 g.L⁻¹ peptone, 10 g.L⁻¹ glucose and 20 g.L⁻¹ Agar, the incubation being run between 24 h and 72 h at temperatures between 25° C. to 30° C.

As fermentation medium are used media containing from 0.01 to 0.5% mass/volume of mineral salts such as K₂HPO₄ and MgSO₄, from 0.1 to 0.5% mass/volume of organic acids such as succinic acid and 0.01% to 1% mass/volume of organic compounds such as soya bran, urea and nitrates.

Useful carbon sources include saccharose, sugar cane molasses, coffee and potato agribusiness, milk serum, besides others.

Other patents, such as U.S. Pat. No. 3,119,812 and U.S. Pat. No. 3,773,752 relate to methods for polymer recovery through-alcohols, with or without salt addition.

The cited patent documents point to the fact that xantan manufacture is based on Xanthomonas campestris. The resulting polymers show the following composition: mannose, glucose and glucuronic acid, besides the pyruvic and acetic radicals.

A drawback exhibited by commercial xantan gums is that in spite of their excellent features, they are unable to yield true gels when used alone, this property being shown only when these products are admixed to galactomannans and glucomannans. Surprisingly, the biopolymers obtained by the present process bear this property: they yield true gels when used alone.

Further, the viscosity of these same state-of-the-art biopolymers does not rise with temperature, this being desirable for several applications. Broadly, the viscosity of all polymers produced by Xanthomonas campestris is reduced as a result of temperature increase. On the contrary, some strains of Xanthomonas arboricola lead to pseudoplastic biopolymers, this being a most relevant technical feature.

Besides, commercial xantan biopolymers show low tolerance to salt addition, even to such low levels as 0.001 to 1% mass/volume (m/v), generally having reduced viscosity for salt additions above 1% m/v, this meaning reduced profits chiefly if the xantan polymer is being used in the petroleum industry or for foodstuff production.

Therefore, in spite of the known developments, the technique still needs a process for producing xantan-like microbian biopolymers based on cultures of Xanthomonas arboricola and/or Xanthomonas arboricola pruni bacterial strains, in fermentation media using residual waters and related products from rice industrial processing and of parboilized rice, where the inoculum is prepared in a medium having a low saccharose or glucose concentration, it being further directed to a liquid fermentation medium containing macro- and micronutrients and other ingredients, the fermentation being conducted under specific process conditions, after which the obtained biopolymers are insolubilized and isolated, such process, the obtained biopolymers, the culture medium and the uses of the biopolymer being described and claimed in the present application.

### SUMMARY OF THE INVENTION

Broadly, the present process for the production of xantan-like biopolymer comprises the steps of:
a) Preparing the initial pre-inoculum by adding to a specific cell growing medium, isolated colonies of Xanthomonas arboricola and/or Xanthomonas arboricola pv pruni grown in a solid medium or lyophilized;
b) Directing the colonies of such initial pre-inoculum to a liquid medium and incubating same for 24 or 48 hours at a temperature between 20° C. to 35° C. and pH 4.5 to 9.0, under agitation of 100 to 250 rpm so as to obtain the final pre-inoculum; the final pre-inoculum can be lyophilized for further use or it can be immediately conveyed to a fermenter;
c) Asseptically conveying the so-obtained final pre-inoculum to a first -sterile fermenter under agitation and aeration, containing liquid medium and saccharose or glucose up to 10% mass/volume based on the medium and incubating for 24 or 48 hours at temperatures between 20° C. to 35° C. under pH 4.5 to 9.0 under agitation between 50 and 1,200 rpm, preferably between 100 to 800 rpm and under aeration by oxygen addition between 0.5 and 4 volumes per volume of air per minute (vvm), preferably between 0.5 and 3.0 vvm so as to obtain the inoculum; the final pre-inoculum,when lyophilized, should be reactivated by resuspending and further incubation under the previous conditions before being transferred to said first fermenter, while the recently prepared final pre-inoculum is directly transferred to said first fermenter;
a) d) Transferring the so-obtained inoculum to a second sterile fermenter, containing the liquid medium for producing the biopolymer by submersed fermentation or by addition of said sterile medium to the inoculum-containing fermenter, fermenting said inoculum under agitation between 50 and 1,200 rpm, preferably between 100 to 800 rpm and under aeration by oxygen addition between 0.5 and 4 volumes per volume of air per minute (vvm), preferably between 0.5 and 3.0 vvm, at a temperature between 22° C. and 35° C. and pH between 4.5 and 9.0 during 24 hours to 120 hours, preferably 48 to 72 hours, the fermentation medium being made up of the soaking or cooking waters of hull-bearing rice or alternatively the water resulting from rice parboilization, besides cellulose, rice and/or wheat bran and/or nitrogen, phosphorus and potassium macronutrients between 0.1 to 4.2 gL⁻¹, and magnesium and iron micronutrients from 0.01 to 1.7 gL⁻¹ and B complex vitamins, Vitamin E, saccharose up to 25% mass/volume and from 50 to 200 ppm silicone oil or rice oil;
e) after the end of the fermentation, filtering the fermented broth for cell separation;
f) inactivating the cells of the fermented broth in the second fermenter itself by thermal sterilization or chemically sterilizing with chlorinated compounds, so as to obtain the desired biopolymer;
g) insolubilizing the obtained biopolymer, optionally after centrifugation, by adding a polar organic solvent preferably an alcohol, to the centrifuged or not broth, added or not of from 0.2 to 10% m/v mono- and/or divalent salts;
h) recovering alcohol solvent by distillation;
i) separating the biopolymer product through draining in a conveyor belt, directing the same to dryers;
j) milling and crushing the biopolymer in a conventional mill; and
k) recovering the ready-to-use xantan biopolymer.

Thus, the invention provides a process for producing a xantan-like biopolymer from Xanthomonas arboricola and/or Xanthomonas arboricola pv pruni cultures, the process involving preparing an inoculum in nutritional media using residual waters and products related to industrial rice processing and parboilized rice.

The invention also provides the xantan-like biopolymer resulting from the said process.

The invention provides further several uses for the so-obtained biopolymer.

The invention provides still the fermentation medium for Xanthomonas arboricola and/or Xanthomonas arboricola pv pruni used for carrying out said process.

The invention further provides the use of a Xanthomonas arboricola and/or Xanthomonas arboricola pv pruni culture for carrying out the process.

### BRIEF DESCRIPTION OF THE DRAWINGS

FIGS. 1 and 2 attached are flow sheets illustrative of the biopolymer production process, FIG. 1 illustrating the invention mode in the presence of cells while FIG. 2 illustrates a process mode for a biopolymer from which the cells have been withdrawn, and then the biopolymer is dried and milled.

FIG. 3 attached is a graph illustrating viscosity values of fermenting broths of two Xanthomonas arboricola strains.

FIGS. 4 and 5 attached are graphs illustrating viscosity values obtained from aqueous biopolymer solutions at 25° C., viscosity data resulting from the same strain at different times (24, 48 and 72 h), at a 1% m/v concentration, and by different Xanthomonas arboricola strains at a 3% m/v concentration. These Figures also illustrate the pseudoplastic behavior of these biopolymer solutions.

FIG. 6 attached is a graph illustrating the viscosity behavior in front of a temperature rise, presented by biopolymers of different Xanthomonas arboricola strains.

FIG. 7 attached is a block diagram illustrating the production interval of the inventive biopolymer by different strain groups, after 72 hours fermentation.

FIG. 8 attached shows the influence or dependence of the aeration condition on biopolymer production. Xantan (g.L⁻¹) process conditions by X. arboricola pv pruni strain 06 in a 3 L capacity fermenter are treatment A (250 rpm and 1.5 volume per volume of air per minute—vvm) and B (350 rpm and 2.0 volume per volume of air per minute—vvm) for agitation and aeration, respectively.

FIG. 9 attached shows the influence of the fermentation period on the apparent viscosity of a biopolymer obtained from the X arboricola pv pruni 06 strain at 6 rpm, for 1% m/v and 2% m/v concentrations.

In FIG. 10 attached a set of graphs illustrates the change in viscosity with the shear rate for xantan-like biopolymers obtained by Xanthomonas arboricola strain 101 in 3% m/v aqueous solutions and added or not added of 1% m/v salts, as compared with commercial xantan polymers. The graphs of FIG. 10 illustrate the compatibility of the biopoymers according to the invention with added salts.

FIG. 11 attached is a set of graphs illustrating the change in viscosity with shear rate for 106 strain or xantan-like biopolymers obtained by Xanthomonas arboricola in 1%mlv aqueous solutions without salt addition and added of 0.1%, 1.0, and 3% m/v salts as compared to a commercial xantan polymer without salt addition.

FIG. 12 attached is a set of graphs illustrating the change in viscosity with shear rate for the 106 strain or xantan-like biopolymers obtained by Xanthomonas arboricola in 1% m/v aqueous solutions without salt addition and added of 0.1%, 1.0, and 3% m/v salts as compared to two commercial xantan polymers to which have been added salts in amounts between 1% m/v and 3% m/v.

FIG. 13 attached is a set of graphs illustrating the change in viscosity with shear rate for Xanthomonas arboricola 06, 101 and 106 strains produced under free and controlled pH, as compared to a commercial xantan gum.

### DETAILED DESCRIPTION OF THE PREFERRED MODES

The invention relates therefore to a process for producing xantan-like microbian biopolymers from cultures of Xanthomonas arboricola and/or Xanthomonas arboricola pv pruni bacterial strains, in fermentation media containing residual waters and products related to the processing of rice and parboilized rice.

Xanthomonas bacteria, belonging to the Pseudomoniaceae family, are Gram-negative, mobile through a single flagellum, strictly aerobic, resistant to streptomycin and essentially phytopathogenic, exception made to Xanthomonas maltophilia. They are widely distributed and infect more than 240 mono- and dicotyledon plant genders. Xanthomonas campestris, the most numerous and abundant, differentiates itself into approximately 125 patovars, those infecting and being the source of diseases in various hosts.

Xanthomonas arboricola pv pruni

Traditionally, xantan producers have used the campestris patovar, more specifically, the NRRL B-1459 and related strains. However, in view of the huge industrial uses of this biopoymer, other potentially xantan-producing microorganisms are being investigated, as well as the optimization of cell growth, production, recovery and purification of the EPS (exopolysaccharides) obtained.

The pruni patovar is the cause of bacterial spots in species of the Prunus (Prunus Bacterial Spots, PBS) gender, such as the peach tree, the almond tree and the plum tree. This disease occurs in all continents and is more serious in areas of hot, humid climate. In a distinguishing way, Xanthomonas campestris shows a systemic activity: it acts throughout the plant, while X pv pruni has a localized action. In the State of Rio Grande do Sul situated in the extreme south of Brazil, this bacterium naturally infects all the cultivated Prunus species, and has been the object of phytopathological studies conducted by the Brazilian State Agricultural Research Company, EMBRAPA-CPACT. To this purpose, more than one hundred strains have already been isolated and identified. However, only recently have the studies for obtaining xantan gum from this patovar started.

There is currently a new classification for the Xanthomonas gender, based on a comparative study of oligonucleotide sequences of the ribosomal nucleic acid (rRNA) or of the sequences of the corresponding genes, and of quantitative DNA homology expressed in hybridization levels of the cell total DNA. According to this new classification, Xanthomonas pruni has been reclassified as Xanthomonas arboricola.

The Applicant notes that all the strains utilized in the development of the present process belong to the EMBRAPA collection of Xanthomonas arboricola and Xanthomonas arboricola pv pruni strains.

A first aspect of the invention is therefore a process for producing xantan-like biopolymers from lyophilized cultures of Xanthomonas arboricola and/or Xanthornonas arboricola pruni bacterial strains.

According to the invention, to effect the present process, lyophilized cultures of Xanthomonas arboricola and/or Xanthomonas arboricola pruni bacterial strains are isolated from necrosed tissues of Prunus gender species, necrosed tissues of hosts having high cellulose content, fruits and leaves of peach tree and plum tree, and fermented in submersed fermentation media, under suitable process conditions to be detailed below in the present specification.

The expression “high cellulose content” means a cellulose content in the range of 38% to 56% based on the total composition.

As most remarkable result of the use of Xanthomonas arboricola and/or Xanthomonas arboricola pv pruni, the xantan biopolymers resulting from the present process differ from the presently commercially available xantan biopolymers in that the presence of rhamnose imparts to such products the ability to form true gels even when used by themselves. As stated hereinbfore, this property is absent from sate-of-the-art xantan biopolymers.

A further advantage is the lower production cost in view of the use of rice processing residual waters, as mentioned hereinbefore.

The major advantage from the use of the present biopolymer is in the petroleum exploration, where high viscosity, temperature resistant polymers are required.

The use in foods is also envisaged as advantageous since the required amount is reduced when compared to that of conventional xantan biopolymers, since the mere presence of salts already existing in product formulations is sufficient to cause a rise in viscosity.

Other uses encompass a paint thickener, as pesticides where it helps in improving adhesion of the pesticide to the plant leaves, avoiding losses of product to the soil and in veterinary products as a vaccine stabilizer.

For a better understanding of the present process, the process steps are described below in detailed form.

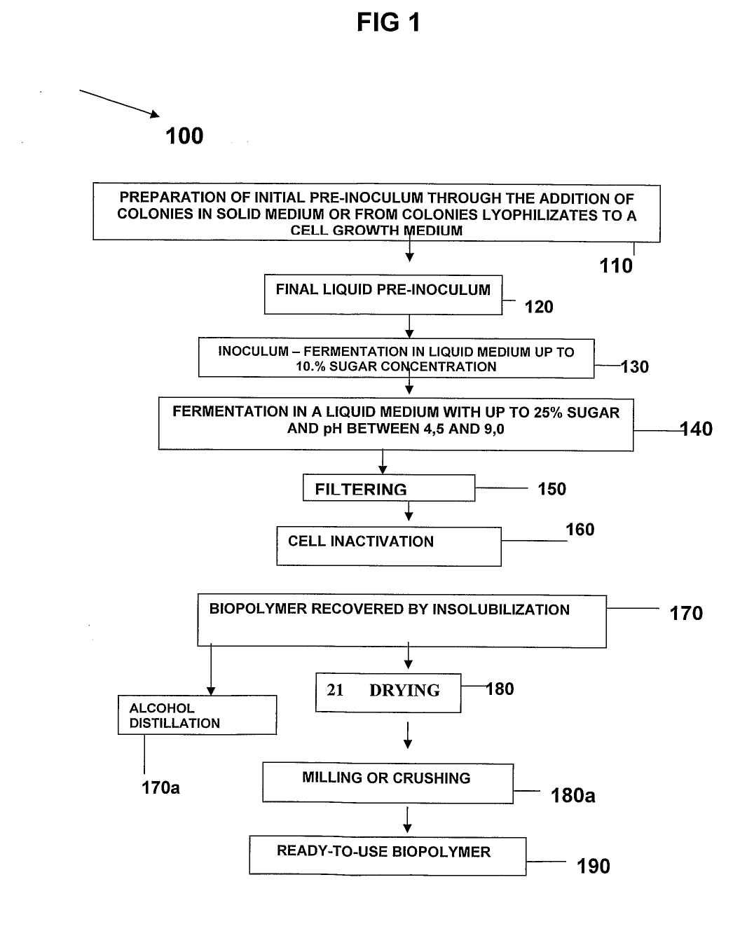

According to the mode of the present process illustrated in the flowsheet of FIG. 1, generally represented by numeral (100), where the second fermentation occurs in a liquid medium of sugar concentration up to 25% mass/volume (or 250 g.L⁻¹) of the fermentation medium, said process comprises the following steps:
a) a) Providing isolated colonies of Xanthomonas arboricola and/or Xanthomonas arboricola pv pruni previously grown in a solid medium or alternatively lyophilized;
b) Preparing the initial pre-inoculum by adding said colonies to a suitable cell growing medium, said medium comprising 13 to 55 gL⁻¹ saccharose or glucose, from 1.0 to 37 g.L⁻¹ peptone, from 10 to 20 g.L⁻¹ Agar, from 0.03 to 0.90g.L⁻¹ K₂HPO₄ and 0.001 to 2.5 g.L⁻¹ MgSO₄ and/or B complex vitamins, the colonies being incubated for 24 h or 48 h under agitation of 100 to 250 rpm at a temperature of 20° C. to 35° C. and pH 4.5 to 9.0 (step 110);

Preferably, the cell growth medium comprises from 10 to 30. gL⁻¹ saccharose or glucose, from 3 to 15 g.L⁻¹ peptone, from 10 to 20 g.L⁻¹ Agar, 0.09 to 0.7 g.L⁻¹ KH₂PO₄ and 0.01 to 1.0 g.L⁻¹ MgSO₄ and/or B complex vitamins. The preferred pH range is between 5.5 to 7.5.
c) Directng the initial pre-inoculum to a liquid medium comprising 13 to 55 g.L⁻¹ saccharose or glucose, 1.0 to 37 g.L⁻¹ peptone, 0.03 to 0.90 g.L⁻¹ K₂HPO₄ and 0.001 to 2.5 g.L⁻¹ MgSO₄ and/or B complex vitamins, the colonies being incubated for 24 h or 48 h at a temperature of 20° C. to 35° C. and pH 4.5 to 9.0, under agitation of 100 to 250 rpm, obtaining after that period the final liquid pre-inoculum, (step 120);

Preferably, the cell growth medium comprises from 10 to 30. gL⁻¹ saccharose or glucose, from 3 to 15 g L⁻¹ peptone, 0.09 to 0.7 g.L⁻¹ KH₂PO₄ and 0.01 to 1.0 g.L⁻¹ MgSO₄ and/or B complex vitamins. The preferred pH range is between 5.5 to 7.5.

The final liquid pre-inoculum can be lyophilized for further use or alternatively be directly transferred to the first fermenter.
d) Asseptically directing the final pre-inoculum to a first sterile fermenter under agitation of 50 to 1,200 rpm, preferably 100 to 800 rpm and aeration by oxygen injection from 0.5 to 4 volume per volume of air per minute, preferably from 0.5 to 3 volume per volume or air per minute, said first fermenter containing a liquid medium made up of up to 100 gL⁻¹ saccharose or glucose, from 1.0 to 37 g.L⁻¹ peptone, from 0.03 to 0.90 g.L⁻¹ K₂HPO₄ and 0.001 to 2.5 g.L⁻¹ MgSO₄ and/or B complex vitamins and incubation for 24 or 48 h, at a temperature of 20° C. to 35° C. and pH from 4.5 to 9.0, obtaining at the end of the fermentation period, the inoculum, (step 130);

The preferred amounts are as for the initial and final pre-inoculums.

Whenever it is lyophilized, the final pre-inoculum should be reactivated by resuspending it and submitting to a fresh incubation under the previous conditions, before its transfer to the first fermenter. On the other hand, the freshly prepared pre-inoculum is directly transferred to a fermenter.
e) Directing the inoculum to a second sterile fermenter, containing the liquid fermentation medium for producing the biopolymer through submerged fermentation or alternatively by adding said sterile medium to the inoculum-containing fermenter, under agitation of 50 to 1,200 rpm, preferably 100 to 800 rpm and aeration by oxygen injection from 0.5 to 4 volume per volume of air per minute, preferably from 0.5 to 3 volume per volume or air per minute, temperature between 22° C. and 35° C., preferably 22° C. and 32° C., pH between 4.5 and 9.0, according to the desired end use for the biopolymer, the fermentation being run for 24 to 120 hours, preferably 48 to 72 hours, the medium being made up of soaking or cooking water of hull-containing rice or the waters resulting from rice parboilization, besides cellulose, rice and/or wheat bran and/or macronutrients nitrogen, phosphorus and potassium from 0.1 to 7.2 g.L⁻¹, and magnesium and iron micronutrients between 0.01 to 1.7 g.L⁻¹ and B complex vitamins, vitamin E saccharose up to 25% mass/volume of liquid medium (or 250 gL⁻¹) and 50 to 200 ppm silicone and/or vegetable oil, (step 140);

Preferably the medium comprises besides the rice waters, macronutrients such as nitrogen, phosphorus and potassium from 1.2 to 4.0 g L⁻¹, as well as micronutrients such as magnesium and iron from 0.1 t o 1.0 g L⁻¹ and B complex vitamins between 0.06 mg L⁻¹ to 2 mg L⁻¹, vitamin E from 10 to 30 μgL⁻¹, saccharose up to 25% mass/volume and from 50 to 200 ppm silicone or rice oil.
e) After the end of the fermentation, filtering the fermented broth for cell separation, step (150);
f) Effecting cell inactivation of the filtered broth in the fermenter itself, through thermal sterilization with live steam at 121° C. or chemical inactivation through the use of chlorinated compounds, step (160);

Thermal sterilization is effected with the aid of live steam, the temperature of which is around 121° C.

Useful chlorinated compounds for chemically inactivating the broth comprise inorganic compounds such as sodium hypochloride and hydrochloric acid used in the concentration between 100 to 200 ppm chlorine. Useful organic compounds include chlorohexidine 0.01 to 0.1% m/v.

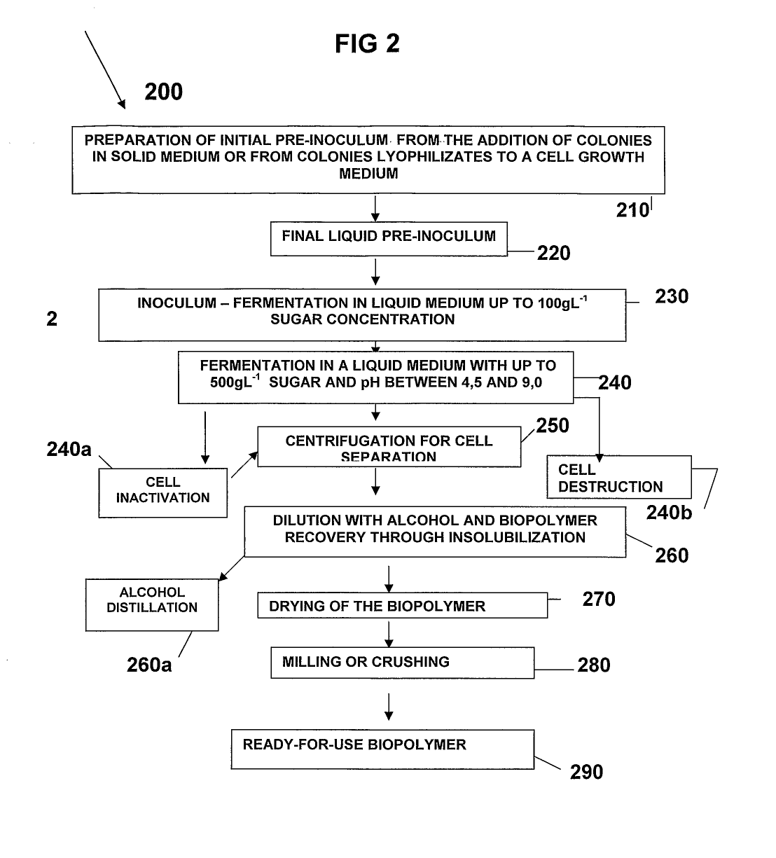

Whenever cell withdrawal is required, insolubilization of the obtained biopolymer is effected after centrifugation (see FIG. 2).

When there is no cell withdrawal, polymer insolubilization is carried out after cell inactivation.

g) Effecting insolubilization, step (170), by addition of polar organic solvent to the inactivated broth, added or not of mono- and/or divalent salts selected among NaCl, KCl and CaCO₃, in concentrations between 0.2 to 10% mass/volume of added solvent;

Polymer precipitation occurs when the solvent concentration attains between 50% to 80% of the total volume. The required solvent volume depends on the added salt percentage.

Useful solvents for the purposes of the invention comprise polar organic solvents, chiefly C₂ and C₃ alcohols, pure or in admixture in any amount.

h) Recovering the polar solvent, such as alcohol, by distillation and re-entry to the process, step (170a);

i) Drying the biopolymer product by initially draining the same in a conveyor belt, then directing the separated product to surface dryers or other similar device, step (180), followed by milling or crushing in any conventional device for this purpose, step (180a); and

f) i) Recovering the xantan-like biopolymer ready for use, step (190).

Optionally, the fermented broth can be directly dried using a spray-dryer or a surface dryer, and then crushing to the desired particle size distribution, in a ball mill or universal mill.

The process is alternatively carried out without pH control (or under free pH conditions), by starting at a nearly neutral pH and letting the reaction system drop the pH to lower values.

Whenever the viscosity of the fermented broth is above 250 mPas at 10 s⁻¹ the same is diluted with water or with a mixture of water and polar organic solvents, preferably C₁ to C₃ alcohols, such as ethyl alcohol and isopropyl alcohol, until the viscosity drops below 250 mPas at 10 s⁻¹. This is an important process feature since too viscous broths mean product loss in the recovery step, which is to be avoided.

Another mode of carrying out the process of the invention is depicted in the flowsheet of FIG. 2, generally represented by numeral (200). According to this mode, a centrifugation step (250) for cell separation and a further step for cell withdrawal or destruction step (260a) are introduced.

According to FIG. 2, step (210) is the preparation of the initial pre-inoculum from the addition to a fermentation medium, of Xanthomonas aroborila and/or Xanthomonas arboricola pv pruni colonies in solid medium or from colonies lyophilizates. Steps (220) and (230) have the same meaning as the corresponding numerals (120) and (130) in FIG. 1.

Step (240) is the second fermentation step, in a liquid medium with up to 500 gL⁻¹ sugar (saccharose or glucose).

The cell inactivation step is designed by step (240a).

A cell destruction step (240b) follows the second fermentation step.

Step (250) relates to a centrifugatin step for cell separation. Centrifugation is effected at 10,000 to 15,000 g.

The centrifuged broth is then submitted to dilution with alcohol (maximum 40% by volume of alcohol) and biopolymer recovery through insolubilization, step (260).

Distillation for recovery of alcohol to be re-used as solvent is step (260a).

After insolubilization the biopolymer product is submitted to the drying (270), milling or crushing (280) and recovery of final biopolymer (290) steps.

The productivity of the bacterial strains used in the present process, in terms of gL⁻¹ of biopolymer obtained attains 5.7 to 26.4, with an average between 15 and 22.

A second aspect of the invention is the fermentation medium used to carry out the 2^{nd} fermentation step of the process for producing the xantan-like biopolymer object of the invention.

Preferred fermentation media are listed below and comprise media numbered A to J.

Medium A.

This fermenation medium comprises:

a) the cooking or soaking waters of hull-containing rice as well as the residual waters of parboilized rice processing (rice parboilization) (also known as soaking waters);

The composition of such rice waters or rice infusion waters includes around 20 mgL⁻¹ to 80 mgL⁻¹ total nitrogen, chiefly as organic nitrogen, this being an excellent substrate for the Xanthomonas pv pruni bacteria. Besides, such water comprises also 10 mgL⁻¹ to 50 mgL⁻¹ phosphate ion and from 2 to 20 mgL⁻¹ sulfate ion.

b) rice bran, commercially available, included in an amount of 0.2 mg to 40 g by L;

c) wheat bran, commercially available, included in an amount of 0.3 gL⁻¹ to 10 gL⁻¹;

d) nitrogen, phosphorus and potassium macronutrients from 0.1 to 7.2 g.L⁻¹, and magnesium and iron micronutrients between 0.01 to 1.7 g.L⁻¹;

e) B Complex vitamins, including vitamins B1, B2 and niacin (vitamin B3) in commercially available, purified form, at concentrations between 0.02 mgL⁻¹ to 3 mgL⁻¹ or alternatively, vitamin B complex-rich natural substrates. Vitamin B complex-rich substrates are those where the sum of these vitamins is higher than 10% mass/mass, such as brewer yeast, yeast extract, dehydrated yeasts, and malt;

g) Vitamin E, included in amount of 10 to 30 μg/L medium or as anti-frothing agent in the course of the process, through the use of vegetable oils rich in this vitamin, such as sunflower oil, cotton oil or soya oil;

h) Sugar as saccharose or glucose in concentration up to 250 g.L⁻¹ or alternatively up to 500 g.L⁻¹.

Alternatively, other media are useful for the production process according to the invention. In some cases using such alternative media rises the process output up to 50%.

Medium B.

The composition of medium B includes in g.L⁻¹: 0.15 to 5.0 KH₂PO₄, 0.01 to 0.6 MgSO₄.7H₂O, 10 to 250 saccharose and 0.2 to 6 rice bran.

Medium C.

The composition of medium C includes, in g.L⁻¹: 0.2 to 1.5g NH₄H₂PO₄; 1 to 5 g K₂HPO₄; 0.1 to 0.6 g MgSO₄.7H₂O, 0.2 to 2.0 citric acid, 2 to 5.0 KH₂PO₄, 0.006 H₃BO₃, 2.0 (NH₄)2SO₄, 0.0024 FeCl₃; 0.002 CaCl₂.2H₂O; 0.002 ZnSO₄, 10 to 250 saccharose, and 0.2 to 6 rice bran.

Media D to J

Other useful fermentation media are listed in Table 1 below, with the composition of different fermentation media having different salt concentrations expressed in g.L⁻¹.

TABLE 1

| | Medium | Medium | Medium | Medium | Medium | Medium | Medium |
|---|---|---|---|---|---|---|---|
| Reactants | D | E | F | G | H | I | J |
| NH₄H₂PO₄ | 1.5 | 1.5 | 0 | 1.5 | 1.5 | 0.75 | 1.5 |
| MgSO₄•7H₂O | 0 | 0.2 | 0.2 | 0.1 | 0.2 | 0.2 | 0.2 |
| K₂HPO₄ | 5.0 | 0 | 5.0 | 5.0 | 2.5 | 5.0 | 5.0 |
| Saccharose | 50.0 | 50.0 | 50.0 | 50.0 | 50.0 | 50.0 | 50.0 |

The invention will be further described in relation to the appended Figures.

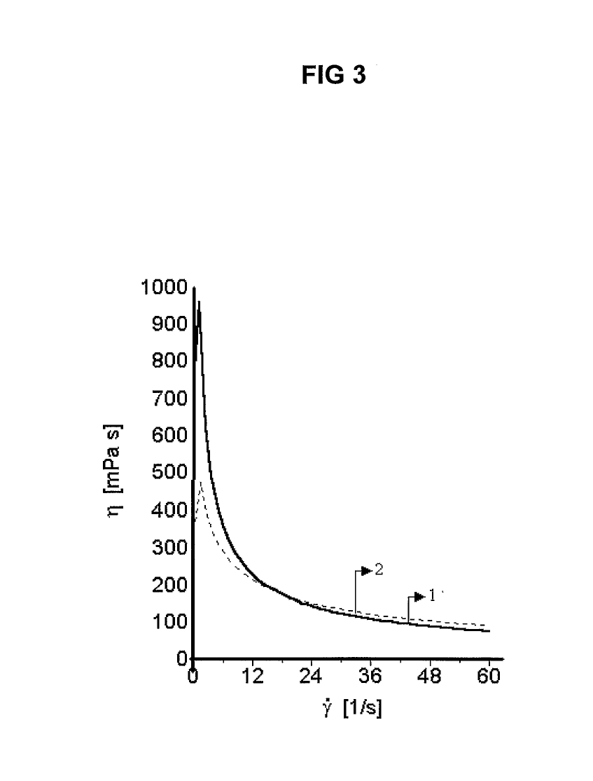

FIG. 3 is a graph illustrative of examples of the viscosity of fermentation media of two Xanthomonas arboricola strains vs. shear rate. The curve of medium 1 relates to strain 82 while curve of medium 2 relates to strain 87.

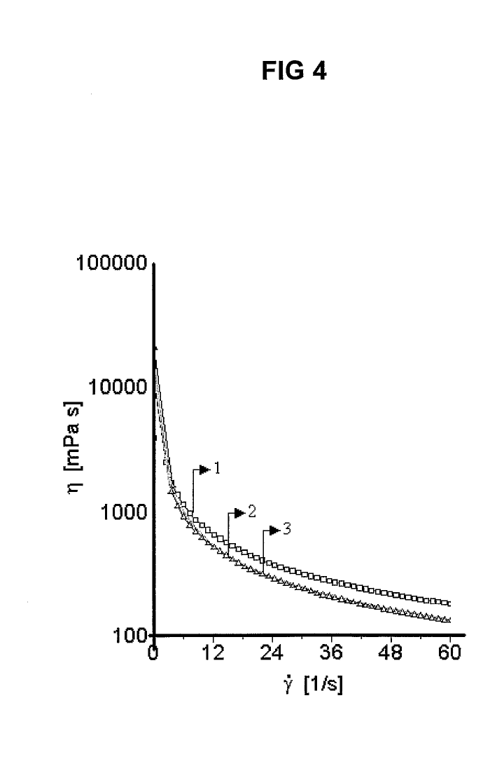

FIG. 4 is a graph illustrative of viscosity values obtained from 1% m/v aqueous solutions of biopolymers at 25° C., produced by one single strain at different times (24, 48 and 72 hours). Curve 1 refers to the biopolymer obrtained after 24 hours, while curves 2 and 3, which are overlapped, refer to the biopolymers obtained after 48 and 72 hours, respectively.

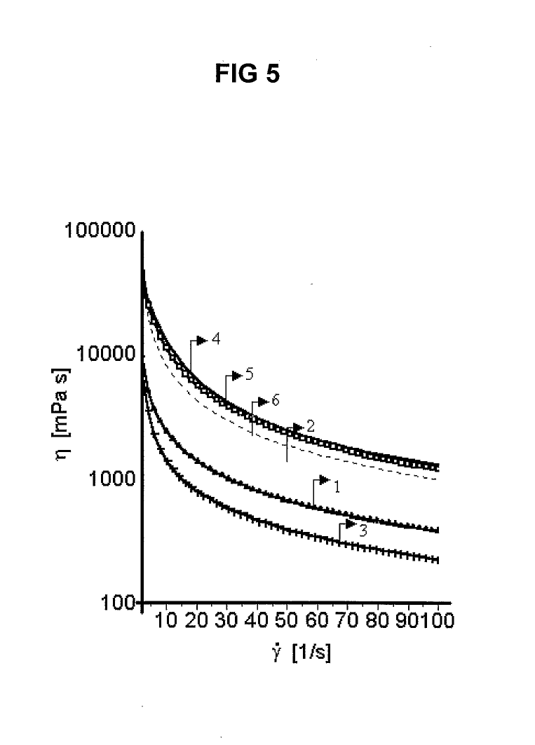

FIG. 5 is a graph illustrative of viscosity values obtained for biopolymers produced by 3% m/v aqueous solutions of different Xanthomonas arboricola strains. Curves 1, 2, 3, 4, 5, 6, are the several viscosity values of biopolymers produced by strains 101, 108, 113, 30, 25, 83, respectively.

FIGS. 4 and 5 also show the pseudoplastic behavior of the tested biopolymer solutions.

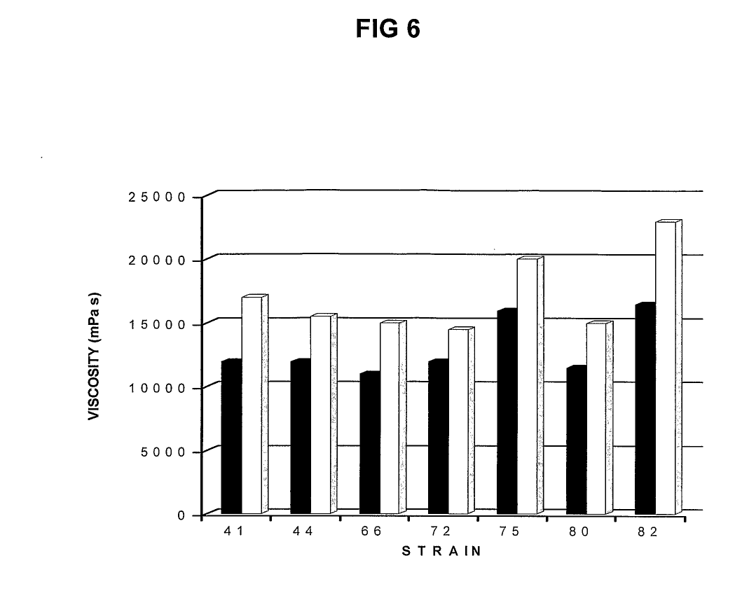

FIG. 6 is a block diagram illustrating the viscosity behavior before the temperature rise shown by biopolymers resulting from different Xanthomonas arboricola strains. In this Figure, full blocks mean viscosity values at 25° C., while empty blocks mean viscosity values at 65° C.

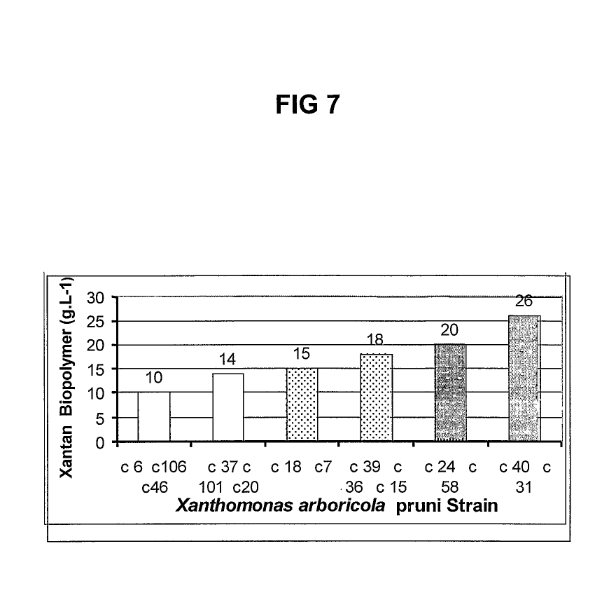

FIG. 7 is a block diagram showing the production interval of the biopolymer of the invention by different groups of strains, after 72 hours fermentation. White blocks represent the production interval comprised between 10 to 14 gL⁻¹ shown by strains 06, 106, 46, 101, 37 and 20. Light-grey blocks represent the production interval comprised between 15 to 18 gL⁻¹ shown by strains 18, 07, 39, 36 and 15. Dark-grey blocks represent the production interval comprised between 20 and 26 gL⁻¹ shown by strains 24, 58, 40 and 31, respectively, obtained in 10 L-capacity fermenters.

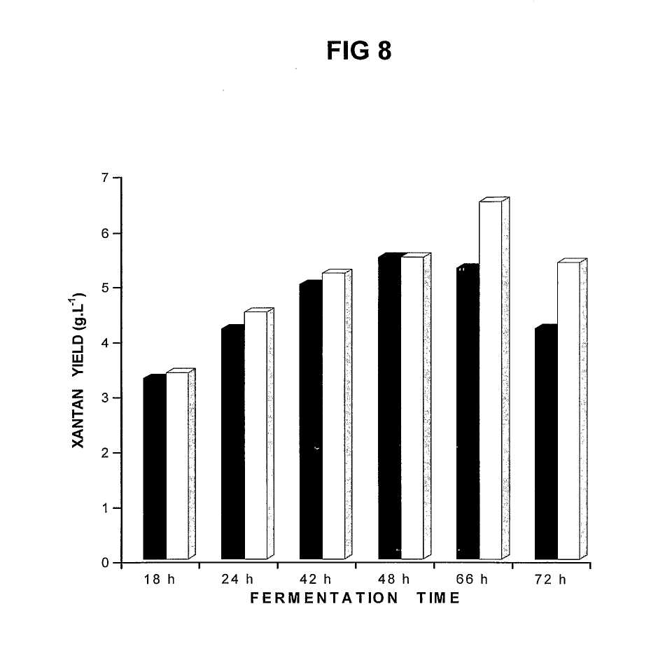

FIG. 8 is a block diagram that shows the influence or dependence on the aeration condition on the xantan biopolymer productivity in gL⁻¹ by strain 06 of Xanthomonas arboricola pv pruni in a 3 L -capacity fermenter. Condition A (250 rpm and 1.5 vvm) for agitation and aeration is represented by full blocks while condition B (350 rpm and 2.0 vvm) is represented by empty blocks.

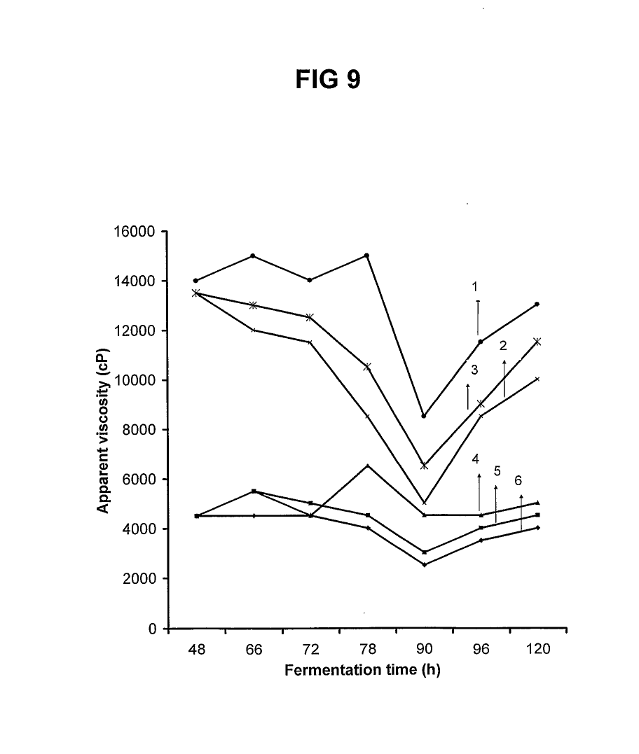

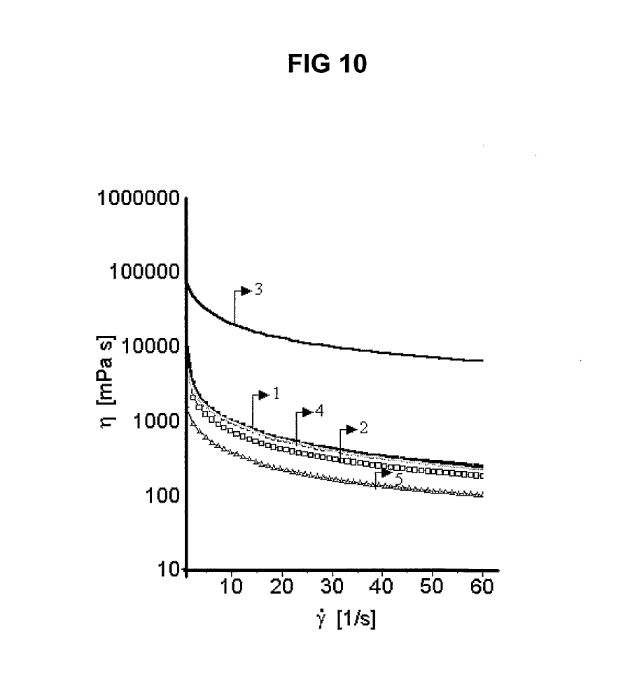

FIG. 9 is a graph illustrating the influence of the fermentation time, of the concentration of biopolymer and of the test temperature on the apparent viscosity of biopolymers produced by strain 06 of Xanthomononas arboricola pv pruni. Curves 1, 2, 3 relate to viscosity values of 2% mass/volume aqueous solutions of biopolymers obtained at different times and measured at 65° C., 45° C., and 25° C. respectively. Curves 4, 5 and 6 relate to viscosity values of 1% mass/volume aqueous solutions of the same biopolymers of curves 1, 2, 3, also measured at the same temperatures.

In FIG. 10 a set of graphs illustrates the change in viscosity with shear rate for xantan-like biopolymers obtained by 1% m/v aqueous solutions of Xanthomonas arboricola strain 101. The solutions optionally are added of 1% or 3% m/v salts, as compared to commercial xantan polymers. The graphs illustrate the compatibility of the biopolymers of the invention with added salts. Curve 1=relates to the viscosity of a commercial xantan biopolymer added of 1% m/v salts. Curve 2 is the same commercial biopolymer added of 3% m/v salts. Curve 3 relates to the biopolymer from strain 101 added of 3% m/v salts, while curve 4 relates to the viscosity of the same biopolymer from strain 101 added of 1% m/v salts. Curve 5 is the viscosity of the biopolymer of strain 101, without any added salts. Note that Curve 3 shows a ten-fold increase in the viscosity of the biopolymer from strain 101 when added of 3% m/v salts as compared to curve 5 that illustrates the same biopolymer without added salts.

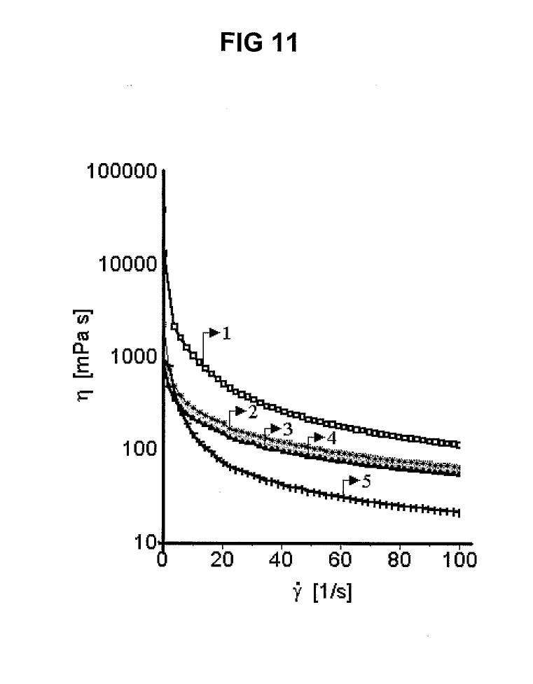

FIG. 11 is a set of graphs illustrating the change in viscosity with shear rate of 1% m/v aqueous solutions of xantan-like biopolymers produced by Xanthomonas arboricola strain 106, with and without thermal inactivation added or not of salts, as compared to a commercial xantan gum to which no salts have been added. Curve 1 relates to the biopolymer obtained by thermal inactivation and added of 1% m/v salts. Curves 2, 3 and 4 relate to the polymer obtained without thermal inactivation and added of 0.1, 1 and 3% m/v salts respectively. Curve 5 realtes to the viscosity of a commercial xantan polymer in aqueous solution, to which no salts have been added.

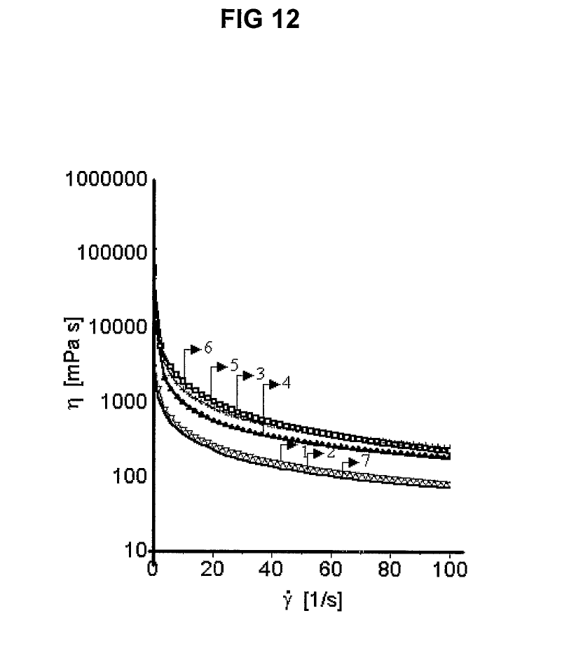

FIG. 12 is a set of graphs illustrating the change in viscosity with shear rate for 106 strain or xantan-like biopolymers obtained by Xanthomonas arboricola in 1% m/v aqueous solutions added of 0.1, 1.0 and 3% m/v salts, as compared to two commercial xantan polymers added of 1% m/v and 3% m/v salts. Curves 1, 2 and 3 relate to the viscosity of strain 106, while 4 and 5 relate to the viscosity of a commercial xantan polymer and curves 6 and 7 of another commercial xantan polymer.

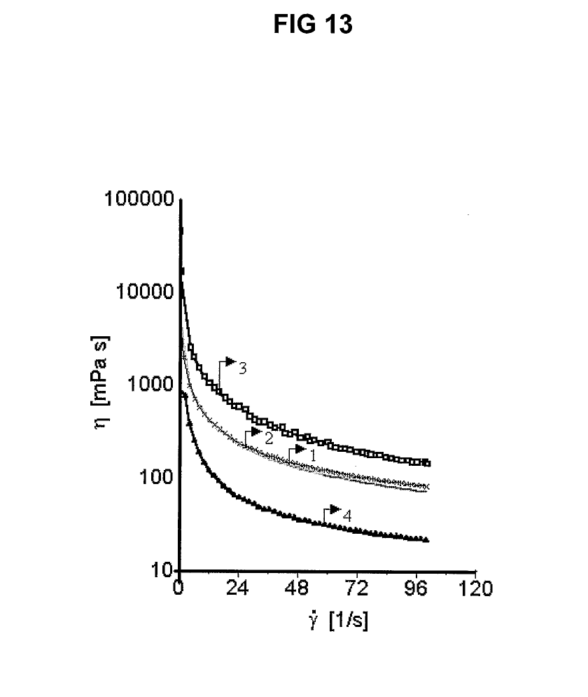

FIG. 13 is a set of graphs illustrating the change in viscosity with-shear rate for xantan-like biopolymers of the Xanthomonas arboricola 06, 106 and 101 strains produced at pH free and controlled, as compared to a commercial xantan polymer. Curve 3 is the viscosity of a 1% m/v aqueous solution of strain 106 biopolymer at a controlled pH of 7, in a 10 L—capacity fermenter, curve 2 is the viscosity of a 1% m/v aqueous solution of strain 06 biopolymer at a controlled pH of 7 and added of 0.1 salts, curve 3 is the viscosity of a 1% m/v aqueous solution of strain 101 biopolymer at a controlled pH of 7 and added of 3% m/v salts, while curve 4 is the 1% m/v viscosity of a commercial xantan biopolymer at 1% m/v salts.

Throughout the present specification the expression Xanthomonas arboricola and/or Xanthomonas arboricola pv pruni is used, meaning that associations of strains are also feasible within the concept of the invention. The only restriction to the admixing of strains is that both should require fermentation conditions including similar process pH range and aeration conditions. Useful combinations are, for example, one strain of relatively low productivity and high viscosity and another strain of higher productivity and not so high viscosity. The association will lead to higher output and higher viscosity than simply the average of both parameters for the two strains.

A third aspect of the invention is the biopolymers obtained through the above-described process.

The features of the inventive biopolymers of Xanthomonas arboricola and/or Xanthomonas arboricola pv pruni resulting from the process comprise:

a—composition, hetero-exopolysaccharide formed chiefly by monosaccharides such as glucose, mannose, glucuronic acid, pyruvic acid, acetic acid and in a distinguishing way relative to Xanthomonas campestris pv campestris and manhiotis, by the presence of rhamnose;

b—high molecular weight, between 4.10⁶ to 12.10⁶ g.mol⁻¹;

c—presentation, the most usable is as a powder, added or not of salts, the biopolymer being easily solubilized in cold or hot water or either in weakly ionic solutions. Alternatively the biopolymer is made available as aqueous concentrated solutions (2 to 6% m/v biopolymer), ready to be added to the products where required;

d—color, as a powder, or even in concentrated solutions, the color varies from light grey to light yellow, seldom reaching dark brown, the exhibited color being a function of the process conditions. By purification the biopolymer is a white or very light yellow product yielding clear solutions even for concentrations as high as 3% m/v or 6% m/v.

The new use of Xanthomonas arboricola and/or Xanthomonas arboricola pv pruni combined to the new fermentation media based on residual waters from rice industries, such as rice parboilization, added of related products and by-products such as rice bran, besides other media cited above, under conditions of aerobic fermentation proposed in the present application makes possible to obtain a new xantan biopolymer.

The chemical composition of the biopolymers produced by Xanthomonas arboricola and/or Xanthomonas arboricola pv pruni is D-mannose, D-glucose, D-glucuronic acid and rhamnose in the amounts: 3:3:1:1, besides acetyl and pyruvic groups in amounts varying from 1.1 to 5.5% and 0.3 to 0.9%, respectively.

As improvement, the biopolymers of the invention are more resistant to temperature than the commercial xantan gums.

The viscosity values of these biopolymers are higher than those of analogous commercial polymers.

Also, the new biopolymers are extremely efficient as regards the salt compatibility, so that it is possible to double viscosity values of 1% m/v and 3% m/v aqueous biopolymer solutions by the addition of 0.2 to 10% m/v salts.

The distinguishing properties of the biopolymers of the invention relative to commercial xantan polymers are illustrated in the Tables below.

Thus, Table 2 below lists values for apparent viscosity vs. temperature resistance of a 3% m/v aqueous solution of biopolymers from Xanthomonas arboricola pv pruni strains at 6 rpm, 25° C. and 65° C. As control, a commercial xantan polymer under the same conditions.

TABLE 2

| Strain number | Viscosity (mPas)at 25° C. | Viscosity (mPas)at 65° C. |
|---|---|---|
| 24 | 25,000 | 19,000 |
| 87 | 22,000 | 15,000 |
| 46 | 23,000 | 21,000 |
| 20 | 15,000 | 15,000 |
| 31 | 20,000 | 21,000 |
| 15 | 34,000 | 38,000 |
| 82 | 15,000 | 22,000 |
| 06 | 27,000 | 32,000 |
| 75 | 15,000 | 19,000 |
| Control | 24,000 | 24,000 |

From the extended experimentation carried out on different strains of Xanthomonas arboricola and Xanthomonas arboricola pv pruni, it could be evidenced that, as shown in Table 2 above, the quality and rheological behavior of the obtaned biopoymer is a function of the specific strain. So, the viscosity of aqueous biopolymers solutions obtained from strains 24, 87 and 46 is reduced as a result of temperature rise, this being a usual feature of biopolymer solutions.

The viscosity values of strains 20 and 31 do not change with the temperature rise, while those of products from strains 15, 82, 06 and 75 undergo a marked increase in viscosity as a result of temperature rise.

Table 3 below ilustrates the influence of the particular strain on the rheological behavior of the biopolymer in aqueous solution. As can be observed from the listed data, the pseudoplasticity shown by biopolymer produced by strain 24 is lower than that shown by commercial xantan polymer and also lower than that of strain 06. The more drastic viscosity reduction means higher pseudoplasticity. Normally higher pseudoplasticity is required for products requiring pumping during processing.

Experimental conditions of tests listed in Table 2 are: 3% m/v aqueous biopolymer solutions, at 25° C and different shear rates, for a commercial Kelco™ product and strains 06 and 24 of Xanthomonas arboricola pv pruni.

TABLE 3

| | Viscosity (mPas) | | |
|---|---|---|---|
| | Kelco ™ xantan | xantan strain 06 | xantan strain 24 |
| Shear rate (s⁻¹) | control | invention | invention |
| 0.005 | 38,000 | 74,000 | 88,000 |
| 10 | 3,300 | 4,040 | 7,000 |
| 30 | 1,300 | 1,550 | 2,640 |
| 60 | 700 | 810 | 1,430 |

Data from Table 3 above inequivocally illustrate the huge versatility of the biopolymers according to the invention. Thus, the behavior of strain 06, showing marked viscosity reduction with shear rate renders it more suitable to be used in petroleum applications, while strain 24, where viscosity drop is not so marked as a consequence of shear, renders it more suitable to be used in foofstuff and cosmetics applications.

Table 4 below shows that viscosity and rheological behavior is a function of the specific strain used to carry out the process. This is seen by comparing the viscosity of a biopolymer obtained from Xanthomonas arboricola pv pruni and a commercial xantan. The conditions used were: apparent viscosity in mPas at 25° C. of a 3% m/v aqueous xantan biopolymer solution synthesized by 15 strains of Xanthomonas arboricola pv pruni, and of the dyalised xantan polymer made by Kelko.

TABLE 4

| Strain | | | | | |
|---|---|---|---|---|---|
| number | 6 s⁻¹ | 10 s⁻¹ | 12 s⁻¹ | 30 s⁻¹ | 60 s⁻¹ |
| 101 | 20,600 | 12,700 | 10,600 | 4,080 | 2,080 |
| 104 | 18,900 | 12,100 | 10,100 | 3,900 | 2,050 |
| 108 | 18,200 | 11,200 | 9,320 | 3,750 | 1,970 |
| 115 | 15,800 | 10,200 | 8,660 | 3,470 | 1,850 |
| 106 | 15,900 | 9,930 | 8,200 | 3,270 | 1,750 |
| 109 | 15,800 | 9,600 | 7,980 | 3,230 | 1,770 |
| 26 | 14,200 | 8,930 | 7,410 | 2,980 | 1,620 |
| 113 | 13,100 | 8,100 | 6,770 | 2,860 | 1,570 |
| 112 | 12,400 | 7,830 | 6,600 | 2,770 | 1,490 |
| 114 | 11,600 | 7,270 | 6,230 | 2,720 | 1,520 |
| 105 | 12,800 | 7,920 | 6,650 | 2,700 | 1,480 |
| 38 | 12,100 | 7,660 | 6,410 | 2,560 | 1,410 |
| 27 | 11,800 | 7,600 | 6,370 | 2,550 | 1,340 |
| 102 | 11,300 | 7,210 | 6,110 | 2,570 | 1,440 |
| 100 | 11,200 | 7,010 | 5,940 | 2,530 | 1,400 |
| Control | 11,700 | 7,120 | 6,080 | 3,120 | 2,030 |

Control: Dyalised commercial xantan gum, marketed by Kelko.

Table 5 below illustrates the influence of the medium composition and of the fermentation reaction time on the viscosity of the obtained biopolymer, measured at two shear rates. The composition containing media B+C means a medium containing equal amounts of both media.

TABLE 5

| R. Time | Productivity (gL⁻¹) | | Viscosity (mPas) | | | |
|---|---|---|---|---|---|---|
| (h) | Medium C | Med. B + C | Medium C | | Medium B + C | |
| 72 | 2.5 | 12.4 | 14,000* | 28** | 15,000* | 30** |
| 96 | 12.2 | 16.4 | 80,000* | 160** | 26,000* | 52** |

*Shear rate 0.5 s⁻¹
**Shear rate 500 s⁻¹

Data from Table 5 indicate that a mixture of media, B+C, brings significant improvement to the productivity for both fermentation periods. However, if viscosity only is sought, then Medium C alone is better.

Table 6 below lists values for apparent viscosity in mPas of 1% m/v aqueous solutions at 25° C. and shear rate 10 s⁻¹, as well as pH values for xantan polymers resulting from the activity of different Xanthomonas arboricola pv pruni strains, in media formulated according to Table 1.

TABLE 6

| | XANTHOMONAS ARBORICOLA STRAIN/pH | | | | | | | | | |
|---|---|---|---|---|---|---|---|---|---|---|
| MEDIA | 06 | pH | 15 | pH | 24 | pH | 31 | pH | 46 | pH |
| F | 3,700 | 5.5 | 6,200 | 6.4 | 3,900 | 6.2 | 6,500 | 6.2 | 6,900 | 7.0 |
| G | 3,000 | 5.5 | 8,300 | 6.5 | 3,900 | 6.3 | 9,400 | 6.5 | 5,900 | 6.5 |
| H | 6,800 | 6.3 | 8,300 | 6.8 | 5,600 | 6.6 | 7,900 | 6.9 | 10,000 | 7.0 |
| I | 6,000 | 5.3 | 10,200 | 5.3 | 5,200 | 5.3 | 10,700 | 5.2 | 5,700 | 5.6 |
| C | 4,400 | 4.9 | 9,400 | 4.6 | 4,400 | 5.0 | 6,600 | 4.5 | 7,600 | 4.6 |

TABLE 7

| Fermentation | free | | | |
|---|---|---|---|---|
| reaction time (h) | pH/gL⁻¹ | pH 5/gL⁻¹ | pH 7/gL⁻¹ | pH 9/gL⁻¹ |
| 24 | 9.2 | 8.8 | 8.9 | 10.0 |
| 48 | 9.1 | 11.6 | 12.5 | 12.5 |
| 54 | 10.3 | 12.3 | 11.2 | 12.0 |
| 66 | 9.4 | 12.3 | 11.7 | 13.9 |
| 72 | 9.8 | 13.2 | 13.3 | 13.3 |

“free pH” means that the fermentation reaction starts at a pH above neutrality and is left to drop without any addition of basic compound to keep it at values higher than 7.0.

Table 8 below shows that, for strain 106, the viscosity of a biopolymer is a function of the agitation condition employed during the process used to obtain it.

TABLE 8

| | Viscosity (mPas) | | |
|---|---|---|---|
| Shear rate s⁻¹ | 10 | 30 | 60 |
| 200 rpm | 600 | 250 | 144 |
| 400 rpm | 1130 | 379 | 207 |

Table 9 below lists, for strain 106, the productivity of an inactivated broth in gL⁻¹ and shows that biopolymer productivity depends on agitation and fermentation reaction time.

TABLE 9

| Fermentation | | | | |
|---|---|---|---|---|
| reaction time (h) | 200 rpm | 200 rpm | 400 rpm | 400 rpm |
| 24 | 7.14 | 6.46 | 8.93 | 8.96 |
| 48 | 8.73 | 8.86 | 12.3 | 12.5 |
| 54 | 9.61 | 9.12 | 11.13 | 13.15 |
| 66 | 10.42 | 10.27 | 11.6 | 13.85 |
| 72 | 10.38 | 10.73 | 13.3 | 16.5 |

Table 10 below illustrates, for strain 106, the influence of the pH of the fermentation medium on the viscosity of the biopolymer product.

TABLE 10

| | Viscosity (mPas) | | |
|---|---|---|---|
| Shear Rate s⁻¹ | 10 | 30 | 60 |
| free pH * | 632 | 274 | 152 |
| pH 5 | 460 | 175 | 95.5 |
| pH 7 | 1130 | 379 | 207 |
| pH 9 | 986 | 343 | 183 |

* free pH = as above

Table 11 below lists the influence of reaction time and aeration conditions of the fermentation medium on the apparent viscosity of the biopolymers produced by strain 06 at different fermentation times, for two different aeration conditions, tested at three shear rates.

TABLE 11

| | | | | | |
|---|---|---|---|---|---|
| A | B | A | B | A | B |

| R. Time | Shear rate | | |
|---|---|---|---|
| (h) | 10 s⁻¹ | 30 s⁻¹ | 60 s⁻¹ |

| | | | | | | |
|---|---|---|---|---|---|---|
| 18 | 760 | 540 | 300 | 220 | 170 | 130 |
| 24 | 770 | 540 | 310 | 220 | 180 | 120 |
| 42 | 710 | 530 | 260 | 210 | 160 | 120 |
| 48 | 640 | 500 | 240 | 210 | 130 | 100 |
| 66 | 640 | 480 | 230 | 200 | 130 | 110 |
| 72 | 610 | 360 | 230 | 160 | 120 | 90 |

Condition:
A 250 rpm 1.5 vvm
B 350 rpm 2.0 vvm

The data provided for in the several Tables of the present specification, as well as the accompanying Figures demonstrate the advantages of the proposed use of the Xanthomonas arboricola and/or Xanthomonas arboricola pv pruni bacteria in producing high viscosity aqueous biopolymers solutions, the biopolymers being utilized as such, isolated or in combination with other biopolymers, or still, added of salts.

Further, the viscosity of the aqueous solutions of the biopolymers object of the invention is higher than that of similar commercial xantan polymers. Advantgeously, the viscosity of the present biopolymers rises as a result of salt addition, even of monovalent salts, at 0.2 to 10% m/v concentration, preferably from 0.5 to 6% m/v, as illustrated in the graphs of FIGS. 10, 11 and 12.

Besides being salt-tolerant products, xantan biopolymers can be added of anti-microbial agents, such as sodium azide, glutaraldehyde, and formaldehyde among others, in order to improve the shelf-life stability of the biopolymer solutions.

Some of these biopolymers obtained from certain strains such as strain 82, 15, 06 and 75, bear the unusual feature of increased viscosity as a result of temperature rise. This behavior is illustrated in FIGS. 4, 5 and 6.

Also, the biopolymers of the invention are able to form true gels when utilized by themselves, or in association with other polymers, the gel strength being improved when the biopolymer is added of divalent salts such as CaCl₂ or CaCO₃,

In a 1% m/v aqueous biopolymer solution the viscosity varies normally between 1,000 to 5,000 mPas at 10s⁻¹ and at 25° C. However values in the range of 100 mPas at 10s⁻¹ at 25° C. are possible, this does not meaning a lower value product, only a product useful for an application different from a thickening agent.

The viscosity values of 3% m/v aqueous biopolymer solutions vary from 4,000 to 28,000 mPa·s at 10s⁻¹ and at 25° C.

A fourth aspect of the invention relates to the uses of the obtained polymer.

The rheological behavior of solutions obtained from the inventive biopolymers is of paramount importance in determining their use. The high pseudoplasticity shown in FIGS. 3 and 4 is a required parameter for the biopolymer to be applied in petroleum exploration activities.

Thus, xanthan gum or biopolymer is used in various aspects of petroleum production, including oil well drilling, by formulating drilling fluids with or without added solids, hydraulic fracturing, workover, as in workover fluids, completion as in formulations, pipeline cleaning, and enhanced oil recovery fluids.

The present biopolymer modifies the rheological properties of aqueous solutions. It imparts desired properties such as stability, improved texture, and controlled release of active ingredients while still being able to reduce ice formation on freezing as well as elimination of syneresis for an annealed formulation. Biopolymer films may be used in food wrapping applications.

It is also useful in the processing of foodstuffs requiring a pumping step, as well as in other industrial activities requiring pumping of solutions.

The quick solubilization in cold or hot water or still in salt solutions or weakly acidic solutions as well as their compability with salts is also relevant for their use in foods or in other uses depending on this feature.

Further uses of the polymer involve pharmacological and cosmetics compositions, besides paints, pesticide compositions and veterinary products.

We claim:

1. A process for preparing a xantan biopolymer, wherein such process comprises the following steps:
a) Providing isolated colonies of Xanthomonas arboricola and/or Xanthomonas arboricola pv pruni previously grown in a solid medium or alternatively lyophilized;
b) Preparing the initial pre-inoculum by adding said colonies to a suitable cell growing medium, said medium comprising 3 to 55 g.L⁻¹ saccharose or glucose, 1.0 to 37 g.L⁻¹ peptone, 10 to 20 g.L⁻¹ Agar, 0,03 to 0.90 g.L⁻¹ K₂HPO₄ and 0.001 to 2.5 g.L⁻¹ MgSO₄ and/or B complex vitamins, the colonies being incubated for 24 h or 48 h under agitation of 100 to 250 rpm at a temperature of 20° C. to 35° C. and pH 4.5 to 9.0 (step 110);
c) Directng the initial pre-inoculum to a liquid medium comprising 3 to 55g.L⁻¹ saccharose or glucose, 1.0 to 37 g.L⁻¹ peptone, 0.03 to 0.90 g.L⁻¹ K₂HPO₄ and 0.001 to 2.5 g.L⁻¹ MgSO₄ and/or B complex vitamins, the colonies being incubated for 24 h or 48 h at a temperature of 20° C. to 35° C. and pH 4.5 to 9.0, under agitation of 100 to 250 rpm, obtaining after that period the final liquid pre-inoculum, (step 120);
d) Asseptically directing the final pre-inoculum to a first sterile fermenter, to carry out fermentation under agitation of 50 to 1,200 rpm, preferably 100 to 800 rpm and aeration by oxygen injection from 0.5 to 4 volume per volume of air per minute, preferably from 0.5 to 3 volume per volume or air per minute, containing a liquid fermentation medium made up of saccharose or glucose kept up to 100 gL⁻¹, from 1.0 to 37 g.L⁻¹ peptone, from 0.03 to 0.90 g.L⁻¹ K₂HPO₄ and from 0.001 to 2.5 g.L⁻¹ MgSO₄ and/or B complex vitamins and incubation for 24 or 48 h at a temperature of 20° C. to 35° C. and pH from 4.5 to 9.0, obtaining at the end of the fermentation period, the inoculum, (step 130);
e) Directing the inoculum to a second sterile fermenter, containing the liquid fermentation medium for producing the biopolymer through submerged fermentation or alternatively by adding said sterile medium to the inoculum-containing fermenter, under agitation of 50 to 1,200 rpm, preferably 100 to 800 rpm and aeration by oxygen injection from 0.5 to 4 volume per volume of air per minute, preferably from 0.5 to 3 volume per volume or air per minute, temperature between 22° C. and 35° C., preferably 22° C. and 32° C., pH between 4.5 and 9.0, alternatively without pH control (free pH) according to the desired end use for the biopolymer, the fermentation being run for 24 to 120 hours, preferably 48 to 72 hours, the medium being made up of soaking or cooking water of hull-containing rice or the waters resulting from rice parboilization, besides cellulose, rice and/or wheat bran and/or macronutrients nitrogen, phosphorus and potassium from 0.1 to 7.2 g.L⁻¹, and magnesium and iron micronutrients between 0.01 to 1.7 g.L⁻¹ and B complex vitamins, vitamin E and/or nicotinamide, saccharose up to 250 g.L⁻¹ and 50 to 200 ppm silicone and/or vegetable oil, (step 140);
e) After the end of the fermentation, filtering the fermented broth for cell separation, step (150);
f) After the end of the fermentation, effecting cell inactivation of the fermented broth in the fermenter itself, through thermal sterilization with live steam at 121° C. or chemical inactivation through the use of chlorinated compounds, step (160);
g) Effecting insolubilization, step (170), by addition of polar organic solvent to the inactivated broth, added or not of mono- and/or divalent salts selected among NaCl, KCl and CaCO₃, in concentrations between 0.2 to 10% mass/volume;
h) Recovering polar solvent by distillation to be recycled to the process, step (170a, 260a);
i) Drying the biopolymer product by initially draining the same in a conveyor belt, then directing the separated product to surface dryers or other similar device, step (180), followed by milling or crushing in any conventional device for this purpose, step (180a); and
i) Recovering the xantan-like biopolymer ready for use, step (190).

2. A process according to claim 1, wherein in steps b), c) and d), the cell growth medium comprises preferably from 10 to 30. gL⁻¹ saccharose or glucose, from 3 to 15 g.L⁻¹ peptone, from 10 to 20 g.L⁻¹ Agar, 0.09 to 0.7 g.L⁻¹ KH₂PO₄ and 0.01 to 1.0 g.L⁻¹ MgSO₄ and/or B complex vitamins, with the preferred pH range between 5.5 and 7.5.

3. A process according to claim 1, wherein the final liquid pre-inoculum is lyophilized for further use or alternatively directly transferred to the first fermenter.

4. A process according to claim 3, wherein prior to use, the lyophilized final pre-inoculum is reactivated by resuspending and submitting it to a fresh incubation under the previous conditions, before its transfer to the first fermenter.

5. A process according to claim 1, wherein alternatively the process is carried out without pH control (or under free pH conditions), by starting at a nearly neutral pH and letting the reaction system drop the pH to lower values.

6. A process according to claim 1, wherein, in order to improve product recovery, whenever the viscosity of the fermented broth is above 250 mPas at 10 s⁻¹ the same is diluted with water or with a mixture of water and polar organic solvents, selected among C₁ to C₃ alcohols, such as ethyl alcohol and isopropyl alcohol, until the viscosity drops below values of 250 mPas at 10 s⁻¹.

7. A process according to claim 1, wherein in step f) the chlorinated compounds for chemically inactivating the broth comprise inorganic compounds such as sodium hypochloride and hydrochloric acid used in the concentration between 100 to 200 ppm chlorine, while organic compounds include chlorohexidine from 0.01 to 0.1% m/v.

8. A process according to claim 1, wherein alternatively a centrifugation step (250) at 10,000 to 15,000 g for cell separation and a further step for cell withdrawal or destruction step (240b) are carried out after the second fermentation.

9. A process according to claim 1, wherein alternatively the second fermentation step (240) is carried out in a liquid fermentation medium containing saccharose or glucose in amounts of up to 500 gL⁻¹ in said medium.

10. A process according to claim 1, wherein the productivity of the bacterial strains in terms of gL⁻¹ of biopolymer obtained attains 5.7 to 26.4, with an average between 15 and 22.

11. A process according to claim 1, wherein the colonies submitted to said process comprise associations of strains yielding synergistic effects, provided such strains require similar fermentation process conditions in terms of pH range and aeration conditions.

12. A fermentation medium designed to be used in the second fermentation step of the process according to claim 1, wherein said medium comprises:
a) the cooking or soaking waters of hull-containing rice as well as the residual waters of parboilized rice processing;
b) rice bran, included in an amount of 0.2 mg to 40 gL⁻¹;
c) wheat bran, included in an amount of 0.3 gL⁻¹ to 10 gL⁻¹;
d) nitrogen, phosphorus and potassium macronutrients from 0.1 to 7.2 g.L⁻¹, and magnesium and iron micronutrients between 0.01 to 1.7 g.L⁻¹;
e) B Complex vitamins, including vitamins B1, B2 and niacin (vitamin B3) in purified form, at concentrations between 0.02 mgL⁻¹ to 3 mgL⁻¹ or alternatively, vitamin B complex-rich natural substrates;
f) Vitamin E, included in amount of 10 to 30 μg/L from vegetable oils;
g)Sugar as saccharose or glucose in concentration up to 250 g.L⁻¹ or alternatively up to 500g.L⁻¹.

13. A medium according to claim 12, wherein the composition of such rice waters or rice infusion waters includes around 20 mgL⁻¹ to 80 mgL⁻¹ total nitrogen, chiefly as organic nitrogen, this being an excellent substrate for the Xanthomonas pv pruni bacteria. Besides, such water comprises also 10 mgL⁻¹ to 50 mgL⁻¹ phosphate ion and from 2 to 20 mgL⁻¹ sulfate ion.

14. A fermentation medium designed to be used in the second fermentation step of the process according to claim 1, wherein the composition of said medium comprises in g.L⁻¹, from 0.15 to 5.0 KH₂PO₄, from 0.01 to 0.6 MgSO₄.7H₂O, from 10 to 250 saccharose and from 0.2 to 6 rice bran.

15. A fermentation medium according to claim 14, wherein alternatively the rice bran is absent from said medium.

16. A fermentation medium designed to be used in the second fermentation step of the process according to claim 1, wherein the composition of said medium comprises, in g.L⁻¹ from 0.2 to 1.5 g NH₄H₂PO₄; from 1 to 5 g K₂HPO₄; from 0.1 to 0.6 g MgSO₄.7H₂O, from 0.2 to 2.0 citric acid, from 2 to 5.0 KH₂PO₄, 0.006 H₃BO₃, 2.0 (NH₄)2SO₄, 0.0024 FeCl₃; 0.002 CaCl₂.2H₂O; 0.002 ZnSO₄, from 10 to 250 saccharose, and 0.2 to 6 rice bran.

17. A fermentation medium according to claim 16, wherein alternatively the rice bran is absent from said medium.

18. Xantan biopolymers produced by Xanthomonas arboricola and/or Xanthomonas arboricola pv pruni, wherein the chemical composition of same comprises D-mannose, D-glucose, D-glucuronic acid and rhamnose in the amounts: 3:3:1:1, besides acetyl and pyruvic groups in amounts varying from 1.1 to 5.5% and 0.3 to 0.9%, respectively.

19. Xantan biopolymers according to claim 18, wherein the molecular weight of same is between 4.10⁶ to 12.10⁶ g.mol⁻¹.

20. Xantan biopolymers according to claim 18, wherein said biopolymers are usable as a powder.

21. Xantan biopolymers according to claim 18, wherein said biopolymers are usable as 2% to 6% mass/volume clear aqueous solutions.

22. Xantan biopolymers according to claim 18, wherein said biopolymers are salt tolerant, with rise of viscosity values of 1% m/v and 3% m/v aqueous biopolymer solutions consequent to the addition of 0.2 to 10% m/v salts.

23. Xantan biopolymers according to claim 18, wherein said biopolymers bear a pseudoplastic behavior when derived from certain strains.

24. Xantan biopolymers according to claim 18, wherein the viscosity of 1% m/v aqueous biopolymer solutions varies between 1,000 to 5,000 mPas at 10 s⁻¹ and at 25° C.

25. Xantan biopolymers according to claim 24, wherein alternatively the viscosity of said solution is in the range of 100 mPas at 10 s⁻¹ at 25° C.

26. Xantan biopolymers according to claim 18, wherein said biopolymers form true gels even when used alone.

27. Xantan biopolymers according to claim 18, wherein said biopolymers are useful in petroleum exploration activities.

28. Xantan biopolymers according to claim 27, wherein such uses include oil well drilling through formulation of drilling fluids with or without added solids, hydraulic fracturing, workover, completion, pipeline cleaning, and enhanced oil recovery fluids.

29. Xantan biopolymers according to claim 18, wherein said biopolymers are useful in the foodstuff industry.

30. Xantan biopolymers according to claim 29, wherein said biopolymers are added to food compositions or either used for food wrapping applications.

31. Xantan biopolymers according to claim 18, wherein said biopolymers are added to pharmaceutical compositions.

32. Xantan biopolymers according to claim 18, wherein said biopolymers are added to cosmetics compositions.

33. Xantan biopolymers according to claim 18, wherein said biopolymers are added to paint compositions.

34. Xantan biopolymers according to claim 18, wherein said biopolymers are added to pesticide compositions.

35. Xantan biopolymers according to claim 18, wherein said biopolymers are added to veterinary compositions, preferably veterinary vaccines.
